(19) United States
(12) Patent Application Publication
(10) Pub. No.: US 2016/0310400 A1
(43) Pub. Date: Oct. 27, 2016

(54) METHOD FOR PREPARING LIPOSOMES OF RETINALDEHYDE OR OTHER PRECURSORS OF RETINOIC ACID AND PRODUCT THUS OBTAINED

(71) Applicant: DERMOPARTNERS, S.L., Valencia (ES)

(72) Inventors: Gabriel SERRANO SANMIGUEL, Valencia (ES); Juan Manuel SERRANO NUÑEZ, Valencia (ES); Maria NAVARRO MOLINER, Valencia (ES)

(21) Appl. No.: 15/105,327

(22) PCT Filed: Dec. 23, 2013
(86) PCT No.: PCT/ES2013/070922

§ 371 Date: Jun. 16, 2016

(51) Int. Cl. A61K 8/67; A61K 8/14; A61K 9/00; A61K 31/11; A61K 31/203; A61Q 19/00; A61K 9/127; A61K 9/08

(52) U.S. Cl. CPC A61K 8/671; A61K 9/1277; A61K 8/14; A61K 9/127; A61K 9/08; A61K 31/11; A61K 31/203; A61Q 19/00; A61K 9/0014; A61K 2800/412; A61K 2800/805

(57) ABSTRACT

Method for preparing liposomes of retinaldehyde or another precursor of retinoic acid for obtaining liposomes that are phospholipidic in nature containing, at least partially, retinaldehyde or another precursor of retinoic acid as an active agent at a ratio of 0.01% to 1%.

### OBJECT OF THE INVENTION

The invention that is proposed relates to a new method for preparing liposomes of retinaldehyde or other precursors of retinoic acid and the resulting product which has an application in the sector of beauty, cosmetic and dermatology centers.

### BACKGROUND OF THE INVENTION

To date, there are different preparations known to contain retinaldehyde or other precursors of retinoic acid as an active agent in their composition. Until now, preparations with concentrations of 1% retinal were not used due to the intense yellow color and the difficulty to solubilize it in any kind of pharmaceutical form.

Some preparations are made with non-phospholipidic liposomes, some with liposomes with cholesterol and others with lamellar emulsions. This is the case, for example, for patents US2003/118616 and WO9631194.

The invention herein described relates to the method for the obtention and to the use of phospholipid liposomes that, while preventing degradation to retinal or protecting another precursor of retinoic acid from external agents they preserve their efficacy over a long period of time. Liposomes have a lipid bilayer similar to that of the stratum corneum, which confers a good affinity with the skin, favoring its penetration and allowing it to work with lower concentrations of the active agent.

This way, in addition to protecting the active agent, the same level of effectiveness as that of higher concentrations is achieved and also the irritation caused by the retinoids applied topically is prevented.

### SUMMARY OF THE INVENTION

The procedure to obtain the product and the product obtained as proposed by the invention consists in a method for obtaining a suspension of phospholipid liposomes with retinaldehyde or another precursor of retinoic acid.

To produce the suspension of liposomes of retinaldehyde, firstly the active agent, retinaldehyde or any precursor of retinoic acid is solubilized in a solvent compatible with the lipids. A derivative of cholic acid or polyoxyethylene 20 sorbitan monolaurate is added to this mixture. The concentration of lecithin may vary from 46 mg/mL to 184 mg/mL and the concentration of cholic acid may vary from 18.3 mg/mL to 73.2 mg/mL. If polyoxyethylene 20 sorbitan monolaurate is used, the quantity used varies from 12.5 mg/mL to 50 mg/mL.

The mixture will be added to an aqueous phase, distilled water or saline in the reaction equipment and it will be homogenized for 30 minutes. This process takes place at a temperature below 35° C. and is carried out under vacuum. The obtained product can be used directly or mixed at a ratio of 3:1 or 1:1 with distilled water or saline and a preservative agent.

The resulting suspension is filtered through a filter of 0.2 microns. This process results in phospholipid liposomes of retinaldehyde that are sized between 100 and 150 nm and with concentrations of retinaldehyde or another precursor of retinoic acid at a ratio of 0.01% to 1% in an appropriate pharmaceutical form.

### PREFERRED EMBODIMENT OF THE INVENTION

As the preferred embodiment of the invention, the method for obtaining the solution of liposomes of retinaldehyde or another precursor of retinoic acid is carried out in the following way:
The active agent, retinal or any precursor of retinoic acid is solubilized in a compatible solvent along with the lipids.
A derivative of cholic acid or polyoxyethylene 20 sorbitan monolaurate is added to this mixture. The concentration of lecithin may vary from 46 mg/mL to 184 mg/mL and the concentration of cholic acid may vary from 18.3 mg/mL to 73.2 mg/mL. If polyoxyethylene 20 sorbitan monolaurate is used, the quantity used varies from 12.5 mg/mL to 50 mg/mL.
The mixture will be added to an aqueous phase, distilled water or saline in the reaction equipment and it will be homogenized for 30 minutes. This process takes place at a temperature below 35° C. and is carried out under vacuum.
The obtained product can be used directly or mixed at a ratio of 3:1 or 1:1 with distilled water or saline and a preservative agent.
The resulting solution is filtered through a filter of 0.2 microns.

The result is a solution that allows preparing cosmetic or dermatological products with concentrations of retinaldehyde or another precursor of retinoic acid at a ratio of 0.01% to 1%.

Having sufficiently described the nature of the present invention, it must be noted that this invention may undergo variations in its components and percentages, provided that said variations do not substantially vary the characteristics that are claimed in the following section:

1. A method for preparing liposomes of retinaldehyde or other precursors of retinoic acid characterized in that it comprises the following steps:
a mixture is prepared by solubilizing an active agent, retinaldehyde or any precursor of retinoic acid in a compatible solvent along with lipids;
a derivative of cholic acid or polyoxyethylene 20 sorbitan monolaurate is added to the mixture;
a product is obtained by adding the mixture to an aqueous phase, distilled water or saline in reaction equipment and homogenizing for 30 minutes at a temperature below 35° C. and under vacuum;
the product is filtered through a filter of 0.2 microns.

2. The method of claim 1, in which the concentration of lecithin of the lipids is in the range of 46 mg/mL to 184 mg/mL.

3. The method of claim 1, in which the concentration of the derivative of cholic acid is in the range of 18.33 mg/mL to 73.2 mg/mL.

4. The method of claim 1, in which the concentration of polyoxyethylene 20 sorbitan monolaurate is in the range of 12.5 mg/mL to 50 mg/mL.

5. A liposome product of retinaldehyde or other precursors of retinoic acid in which the liposomes are phospholipidic in nature, prepared by a method comprising the steps of:
preparing a mixture by solubilizing an active agent, retinaldehyde or any precursor of retinoic acid in a compatible solvent along with lipids;
a derivative of cholic acid or polyoxyethylene 20 sorbitan monolaurate is added to the mixture:
a product is obtained by adding the mixture to an aqueous phase, distilled water or saline in reaction equipment and homogenizing for 30 minutes at a temperature below 35° C. and under vacuum;
the product is filtered through a filter of 0.2 microns.

6. The liposome product of claim 5, in which the product contains, at least partially, retinaldehyde as an active agent, at a concentration of 0.01% to 1%.

7. The liposome product of claim 5, in which the product contains, at least partially, other precursors of retinoic acid as an active agent, at a concentration of 0.01% to 1%.

8. The method of claim 1 in which the product is mixed at a ratio of 3:1 or 1:1 with distilled water or saline and a preservative agent.

9. The liposome product of claim 5, in which the concentration of lecithin of the lipids is in the range of 46 mg/mL to 184 mg/mL.

10. The liposome product of claim 5, in which the concentration of the derivative of cholic acid is in the range of 18.33 mg/mL to 73.2 mg/mL.

11. The liposome product of claim 5, in which the concentration of polyoxyethylene 20 sorbitan monolaurate is in the range of 12.5 mg/mL to 50 mg/mL.
